SEQUENCE LISTING (57 sequences)

SEQ ID NO: 1 - DNA, 4345 bp, H. influenzae
  CDS (403)..(849)
gcgatcattaaaattgacatattgcgtaattcgcccatttcgttcgatcaaacaatgtgctgaaacacgcatttgataaatttctgcaaaataaggatgaatcttaggatctaattttccttgaaaaaaatcatccacatatccgccgccaaattgttctggcggcagactaatataatgaataaccaataaggaaatatcctgtggatttgggcgtttatcgaagtgaggtgactgaatttgccgacaatccaatataccttgttcaatatcttttagtttttgcatacttttttcctttttttgcgatcaggatcgcagaaaaagtgcggtcaattttacaaacaaatttttccttttcacaatgtcgtcgctaacaaaggcttaataaaaggaaaatgaatgaaactaacaacacagcaaaccttgaaaaaagggtttacattaatagagctaatgattgtgattgcaattattgctattttagccactatcgcaattccctcttatcaaaattatactaaaaaagcagcggtatctgaattactgcaagcgtcagcgccttataaggctgatgtggaattatgtgtatatagcacaaatgaaacaacaaactgtacgggtggaaaaaatggtattgcagcagatataaccacagcaaaaggctatgtaaaatcagtgacaacaagcaacggtgcaataacagtaaaaggggatggcacattggcaaatatggaatatattttgcaagctacaggtaatgctgcaacaggtgtaacttggacaacaacttgcaaaggaacggatgcctctttatttccagcaaatttttgcggaagtgtcacacaatgacgagctatgctttacttcatactcagcgtgtaaccgctcaaaatggcgagatctttacgatctcgccagatttatgggaacgcaatcagcagcaacaatccttgctcttgcggtattttgctttgccacttaaagaagaaaataatcgtctttggctaggggttgattctctctccaatctttcagcttgtgaaaccattgcgtttataacaggaaaacctgtcgaaccaattttgttagaaagcagccaactcaaagaactgttacaacaacttactccgcaccaaatgcaagtggaagagcaagttaaattctatcaacatcaagaaacccattttgaacaagaagatgatgaacctgttatccgcttacttaatcagatttttgaatctgccttacaaaaaaatgcctctgatattcatttagaaaccttggctgatcagtttcaagtgcggtttagaattgatggtgttttacaaccacaacccttaataagcaaaatattcgccaatcgtattatttcacgcttaaaattactggctaaattagatattagtgaaaatcgacttccacaagatggacgatttcaatttaaaaccactttttccgatattcttgattttcgcctttcaaccttaccaacccattggggcgaaaaaatcgtgttgcgagcgcaacaaaataaacctgtagaacttagctttgctgaactgggtatgaccgaaaatcagcaacaagcatttcaacgctcacttagccagccacaaggattaattttagtaaccggccccacaggaagtgggaaaagtatctcgctttacaccgcacttcagtggctaaatacgcctgataaacatattatgaccgctgaagatcccattgaaattgaacttgatggtattattcaaagccaaattaatccgcagattggattagattttagccgtctattgcgtgcttttttacgtcaagatcccgacatcattatgctaggtgaaattcgagatgaagaaagtgcaaggattgcactacgtgccgctcaaacgggacatttggtgctttcaactttacataccaatgatgcaatatctgccatttctcgcttacaacaactcggtattcaacaacatgaaattgaaaacagtttactactcgtcattgcacagcgtcttgtacgaaaaatctgtccaaagtgcggtggaaatttaataaattcttgtgattgccatcaaggttatcgagggcgaatcggcgtgtatcaatttctacattggcaacagaatggctatcaaacggattttgagaatttacgagagagtggtttggaaaaagttagccaaggcataacagatgagaaagaaattgaacgtgtgttaggtaaaaactcatgactaaaaaactcttttattatcaaggtagtaacgcattaaatcagaaacaaaaaggctcaattattgcggatacgaaacaacaagcgcactttcagttaataagccgcgggcttactcacatcaaattacaacaaaactggcaatttggggcaaaacccaaaaattcagaaatcagtgaattactcaatcaattagcgacattgctacagtccgtaattccgttaaaaaacagcctacaaattttgcaacaaaattgtactcaaattatgctcaacaaatggcttgaacgactgcttcaatccattgaatctggcttagcattctcacaagccattgaacaacaaggaaaatatctcacacaacaagaaattcaactgattcaagtgggagaaatgacaggaaaacttgccgtagtttgtaaaaaaatagccacgcaccgtagtcaatctttggctttacaacgcaaattacagaaaattatgttatatccctcaatggtattgggaatttctctattattgacactcgcattactgctttttatcgcgcctcaatttgctgaaatgtacagtggcaataatgcggagttaccaacaataaccgcaatattgctctcaatatctaatttccttaagcaaaatattggcattttgctatttttcgttttgagtttttttctattttattatttctatctaaaacgccagacttggtttcatcaaaagaaaaatcaacttatttctatcacgcctatttttggcacaattcaaaagctttcacgtttagtgaactttagtcaaagtttacaaattatgttgcaggccggcgtaccgcttaatcaggcactagacagttttcttcctcgcacacaaacttggcaaaccaagaaaacgcttgtaaacgatatggtattagataaagaagtgcggtcaattttgcaatgggtttctcaaggctatgcgttttctaatagcgtaagtagcgatcttttcccgatggaagcacaacaaatgctacaaattggcgaacaaagcggaaaactcgctttgatgctagagcatatcgcagataattaccaagaaaaacttaatcatcaaattgacttactctcacaaatgctagaaccattaatgatggtaatcatcggcagtctgattgggattattatgatgggaatgtatttacctatctttaatatgggatcagttattcaatgatttacttcacaatgtttttattaggcggcatcttagggatcgcattgtggttctacctatctggttttattacgcatttgcagcaagagatttatgcgacttacgttgaattatttccacaaaacagttctccatttcaaccgcactttgcctctattcaacaaaaaaagtgcggtcatattttgaggtatttttttagtattggggttggatttatatttttacaaattgccttcaaagattctatttttactgtatggatcggactcacacttattattctttggacaatcagttatcttgattggcactatcaacttatttctacgacaccctgtttatggttacttactctcggtttatttggcgcagacaataacttttcattgctaacgttatctgaaagcataaaaagtgcggctagtttttttattgttttctacgcaatctattggattgcaaaatgttattatagaaaagaagcctttggacggggagattattggctagcaatggcattaggaagttttattcatttagaaaccttaccgcactttttattattagcctcagtgcttggaatatgtttttcgcttattcataaaaagaaaaaagaatttataccttttgccccttttatgaacttatcggctatcattatttatctcgtcaaatattacggatattaaaaaggggaaaacataatatttttcccttgttcttcatagaagtgcggttgtttttacgaacgtttcatcacttcaaaaaactcttcgttggttttcgccatcatcagcttatcaatcaagaattccattgcatccacttcatccattggattaagaatcttacgaagaatccacattttttgtaattcgtccgctg

SEQ ID NO: 2 - protein, 149 aa, H. influenzae
MKLTTQQTLKKGFTLIELMIVIAIIAILATIAIPSYQNYTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 3 - protein, 131 aa, H. influenzae
MGSSHHHHHHSSGLVPRGSHMTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 4 - protein, 19 aa, Artificial sequence
  Synthetic Peptide
RSDYKFYEDANGTRDHKKG

SEQ ID NO: 5 - protein, 24 aa, Artificial sequence
  Synthetic Peptide
LVRSDYKFYEDANGTRDHKKGRHT

SEQ ID NO: 6 - protein, 22 aa, Artificial Sequence
  Synthetic Peptide
RSDYKLYNKNSSSNSTLKNLGE

SEQ ID NO: 7 - protein, 19 aa, Artificial sequence
  Synthetic Peptide
RSDYKLYNKNSSTLKDLGE

SEQ ID NO: 8 - protein, 13 aa, Artificial sequence
  Synthetic Peptide
RSDYKFYDNKRID

SEQ ID NO: 9 - protein, 139 aa, Artificial sequence
  Chimeric protein
MGSSHHHHHSSGLVPRGSHMTKKAAVSELLQASAPYKADVELCRSDYKFYEDANGTRDHKKGCTGGKNGIAADITTAKGYVKSVTTSNGAITVKGDGTLANMEYILQATGNAATGVTWTTCKGTDASLFPANFCGSVTQ

SEQ ID NO: 10 - protein, 143 aa, Artificial sequence
  Chimeric Protein
MGSSHHHHHSSGLVPRGSHMTKKAAVSELLQASAPYKADVELCLVRSDYKFYEDANGTRDHKKGHTCTGGKNGIAADITTAKGYVKSVTTSNGAITVKGDGTLANMEYILQATGNAATGVTWTTCKGTDASLFPANFCGSVTQ

SEQ ID NO: 11 - protein, 136 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCRSDYKFYEDANGTRDHKKGCGSVTQ

SEQ ID NO: 12 - protein, 141 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCLVRSDYKFYEDANGTRDHKKGRHTCGSVTQ

SEQ ID NO: 13 - protein, 148 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCKGTDASLFPANFCGSVTQRSDYKFYEDANGTRDHKKG

SEQ ID NO: 14 - protein, 153 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCKGTDASLFPANFCGSVTQLVRSDYKFYEDANGTRDHKKGRHT

SEQ ID NO: 15 - protein, 142 aa, Artificial sequence
  Chimeric Protein
MGSSHHHHHSSGLVPRGSHMTKKAAVSELLQASAPYKADVELCRSDYKLYNKNSSSNSTLKNLGECTGGKNGIAADITTAKGYVKSVTTSNGAITVKGDGTLANMEYILQATGNAATGVTWTTCKGTDASLFPANFCGSVTQ

SEQ ID NO: 16 - protein, 147 aa, Artificial sequence
  Chimeric Protein
MGSSHHHHHSSGLVPRGSHMTKKAAVSELLQASAPYKADVELCLVRSDYKLYNKNSSSNSTLKNLGERHTCTGGKNGIAADITTAKGYVKSVTTSNGAITVKGDGTLANMEYILQATGNAATGVTWTTCKGTDASLFPANFCGSVTQ

SEQ ID NO: 17 - protein, 139 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCRSDYKLYNKNSSSNSTLKNLGECGSVTQ

SEQ ID NO: 18 - protein, 144 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCLVRSDYKLYNKNSSSNSTLKNLGERHTCGSVTQ

SEQ ID NO: 19 - protein, 151 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCKGTDASLFPANFCGSVTQRSDYKLYNKNSSSNSTLKNLGE

SEQ ID NO: 20 - protein, 156 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCKGTDASLFPANFCGSVTQLVRSDYKLYNKNSSSNSTLKNLGERHT

SEQ ID NO: 21 - protein, 139 aa, Artificial sequence
  Chimeric Protein
MGSSHHHHHSSGLVPRGSHMTKKAAVSELLQASAPYKADVELCRSDYKLYNKNSSSLKNLGECTGGKNGIAADITTAKGYVKSVTTSNGAITVKGDGTLANMEYILQATGNAATGVTWTTCKGTDASLFPANFCGSVTQ

SEQ ID NO: 22 - protein, 145 aa, Artificial sequence
  Chimeric Protein
MGSSHHHHHSSGLVPRGSHMTKKAAVSELLQASAPYKADVELCLVRSDYKLYNKNSSSTLKNLGERHTCTGGKNGIAADITTAKGYVKSVTTSNGAITVKGDGTLANMEYILQATGNAATGVTWTTCKGTDASLFPANFCGSVTQ

SEQ ID NO: 23 - protein, 136 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCRSDYKLYNKNSSTLKNLGECGSVTQ

SEQ ID NO: 24 - protein, 141 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCLVRSDYKLYNKNSSTLKNLGERHTCGSVTQ

SEQ ID NO: 25 - protein, 148 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCKGTDASLFPANFCGSVTQRSDYKLYNKNSSTLKNLGE

SEQ ID NO: 26 - protein, 153 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCKGTDASLFPANFCGSVTQLVRSDYKLYNKNSSTLKNLGERHT

SEQ ID NO: 27 - protein, 133 aa, Artificial sequence
  Chimeric Protein
MGSSHHHHHSSGLVPRGSHMTKKAAVSELLQASAPYKADVELCRSDYKFYDNKRIDCTGGKNGIAADITTAKGYVKSVTTSNGAITVKGDGTLANMEYILQATGNAATGVTWTTCKGTDASLFPANFCGSVTQ

SEQ ID NO: 28 - protein, 138 aa, Artificial sequence
  Chimeric Protein
MGSSHHHHHSSGLVPRGSHMTKKAAVSELLQASAPYKADVELCLVRSDYKFYDNKRIDRHTCTGGKNGIAADITTAKGYVKSVTTSNGAITVKGDGTLANMEYILQATGNAATGVTWTTCKGTDASLFPANFCGSVTQ

SEQ ID NO: 29 - protein, 130 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCRSDYKFYDNKRIDCGSVTQ

SEQ ID NO: 30 - protein, 135 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCLVRSDYKFYDNKRIDRHTCGSVTQ

SEQ ID NO: 31 - protein, 142 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCKGTDASLFPANFCGSVTQRSDYKFYDNKRID

SEQ ID NO: 32 - protein, 147 aa, Artificial sequence
  Chimeric Protein
MGSSHHHHHSSGLVPRGSHMTKKAAVSELLQASAPYKADVELCVTSTNETTNCTGGKNGIAADITTAKGYVKSVTTSNGAITVKGDGTLANMEYILQATGNAATGVTWTTCKGTDASLFPANFCGSVTQLVRSDYKFYDNKRIDRHT

SEQ ID NO: 33 - DNA, 447 bp, H. influenzae
  CDS (1)..(447)
atgaaattaacaacacagcaaaccttgaaaaaagggtttacattaatagagctaatgattgtgattgcaattattgctattttagccactatcgcaattccctcttatcaaaattatactaaaaaagcagcggtatctgaattactgcaagcgtcagcgccttataaggctgatgtggaattatgtgtatatagcacaaatgaaacaacaaactgtacgggtggaaaaaatggtattgcagcagatataaccacagcaaaaggctatgtaaaatcagtgacaacaagcaacggtgcaataacagtaaaaggggatggcacattggcaaatatggaatatattttgcaagctacaggtaatgctgcaacaggtgtaacttggacaacaacttgcaaaggaacggatgcctctttatttccagcaaatttttgcggaagtgtcacacaa

SEQ ID NO: 34 - protein, 149 aa, H. influenzae; identical to SEQ ID NO: 2

SEQ ID NO: 35 - DNA, 447 bp, H. influenzae; identical to SEQ ID NO: 33
  CDS (1)..(447)

SEQ ID NO: 36 - protein, 149 aa, H. influenzae; identical to SEQ ID NO: 2

SEQ ID NO: 37 - DNA, 447 bp, H. influenzae; identical to SEQ ID NO: 33
  CDS (1)..(447)

SEQ ID NO: 38 - protein, 149 aa, H. influenzae; identical to SEQ ID NO: 2

SEQ ID NO: 39 - DNA, 447 bp, H. influenzae; identical to SEQ ID NO: 33
  CDS (1)..(447)

SEQ ID NO: 40 - protein, 149 aa, H. influenzae; identical to SEQ ID NO: 2

SEQ ID NO: 41 - DNA, 447 bp, H. influenzae
  CDS (1)..(447)
atgaaattaacaacacagcaaaccttgaaaaaaggttttacattaatcgagctaatgattgtgattgcaattattgctattttagccactatcgcaattccttcttatcaaaattataccaaaaaagcttcggtatccgaattactgcaagcatctgcaccttataaggctgatgtggaattatgtgtatatagcacaaatgaaacaacaaactgtacgggtggaaaaaatggtattgcagctgatattacaacagcaaaaggctatgtagcctcagtgaaaactcaatcgggtggcattacagtaaaagggaatggcacattggcaaatatggaatatattttgcaagctaaaggtaatgctacagcaggtgtaacttggacaacaacctgcaaaggaacggatgcctctttatttccagcaaatttttgcagaagtgtcacaaaa

SEQ ID NO: 42 - protein, 149 aa, H. influenzae
MKLTTQQTLKKGFTLIELMIVIAIIAILATIAIPSYQNYTKKASVSELLQASAPYKADVELCVYSTNETTNCTGGKNGIAADITTAKGYVASVKTQSGGITVKGNGTLANMEYILQAKGNATAGVTWTTTCKGTDASLFPANFCRSVTK

SEQ ID NO: 43 - DNA, 447 bp, H. influenzae
  CDS (1)..(447)
atgaaattaacaacacagcaaaccttgaaaaaagggtttacattaatagagctaatgattgtgattgcaattattgctattttagccaccatcgcaattccttcttataaaaattatactaaaaaagcagcggtatctgaattactgcaagcttctgcgccttataaggctgatgtggaattatgtgtatatagcacaaatgaaataacaaattgtatgggtggaaaaaatggtattgcagctgatattacaacagcaaaaggctatgtagcctcagtgaaaactcaatcgggtggcattaccgtaaaaggggatggcacattggcaaatatggaatatattttgcaagctacaggtaatgctgcagcaggtgtaacttggacaacaacctgcaaaggaacggatgcctctttatttccagcaaatttttgcggaagtatcacacaa

SEQ ID NO: 44 - protein, 149 aa, H. influenzae
MKLTTQQTLKKGFTLIELMIVIAIIAILATIAIPSYKNYTKKAAVSELLQASAPYKADVELCVYSTNEITNCMGGKNGIAADITTAKGYVASVKTQSGGITVKGDGTLANMEYILQATGNAAAGVTWTTTCKGTDASLFPANFCGSITQ

SEQ ID NO: 45 - DNA, 447 bp, H. influenzae
  CDS (1)..(447)
atgaaattaacaacactgcaaaccttgaaaaaagggtttacattaatcgagctaatgattgtgattgcaattattgctattttagccactatcgcaattccttcttatcaaaattataccaaaaaagctgcggtatccgaattactgcaagcttctgcgccttataaggctgatgtggaattatgcgtttatagcacaggcaaaccttctacttgctcaggaggaagcaatggaattgcagctgatattacgacagcaaaaggctatgtagcctcagtgaaaactcaatcaggtggtattacagtaaaagggaatggcacattggcaaatatggaatatattttgcaagctacaggtaatgctgcaacaggtgtaacttggacaacaacttgcaaaggaacggatgcctctttatttccagcaaatttttgcggaagtgtcacacaa

SEQ ID NO: 46 - protein, 149 aa, H. influenzae
MKLTTLQTLKKGFTLIELMIVIAIIAILATIAIPSYQNYTKKAAVSELLQASAPYKADVELCVYSTGKPSTCSGGSNGIAADITTAKGYVASVKTQSGGITVKGNGTLANMEYILQATGNAATGVTWTTTCKGTDASLFPANFCGSVTQ

SEQ ID NO: 47 - DNA, 447 bp, H. influenzae
  CDS (1)..(447)
atgaaattaacaacactgcaaaccttgaaaaaagggtttacattaatcgagctaatgattgtgattgcaattattgctattttagccactatcgcaattccttcttatcaaaattataccaaaaaagctgcggtatccgaattactgcaagcttctgcgccttataaggctgatgtggaattatgcgtttatagcacaggcaaactttctacttgctcaggaggaagcaatggaattgcagctgatattacaacagcaaaaggctatgtagcctcagtgaaaactcaatcaggtggtattacagtaaaagggaatggcacattggcaaatatggaatatattttgcaagctaaaggtaatgctacagcaggtgtaacttggacaacaacctgcaaaggaacggatgcctctttatttccagcaaatttttgcggaagtgtcacaaaa

SEQ ID NO: 48 - protein, 149 aa, H. influenzae
MKLTTLQTLKKGFTLIELMIVIAIIAILATIAIPSYQNYTKKAAVSELLQASAPYKADVELCVYSTGKLSTCSGGSNGIAADITTAKGYVASVKTQSGGITVKGNGTLANMEYILQAKGNATAGVTWTTTCKGTDASLFPANFCGSVTK

SEQ ID NO: 49 - DNA, 447 bp, H. influenzae
  CDS (1)..(447)
atgaaattaacaacacagcaaaccttgaaaaaaggttttacattaatcgagctaatgattgtgattgcaattattgctattttagccactatcgcaattccttcttatcaaaattataccaaaaaagcttcggtatccgaattactgcaagcttccgcaccttataagtcagatgtggaattatgcgtttatagcacaggcaaaccttctacttgctcaggaggaagcaatggaattgcagctgatattacaacagcaaaaggctatgtagcctcagtgaaaactcaatcaggtggtattacagtaaaagggaatggcacattggcaaatatggaatatattttgcaagctaaaggtaatgctacagcaggtgtaacttggacaacaacctgcaaaggaacggatgcctctttatttccagcaaatttttgcagaagtgtcacaaaa

SEQ ID NO: 50 - protein, 149 aa, H. influenzae
MKLTTQQTLKKGFTLIELMIVIAIIAILATIAIPSYQNYTKKASVSELLQASAPYKSDVELCVYSTGKPSTCSGGSNGIAADITTAKGYVASVKTQSGGITVKGNGTLANMEYILQAKGNATAGVTWTTTCKGTDASLFPANFCRSVTK

SEQ ID NO: 51 - DNA, 447 bp, H. influenzae
  CDS (1)..(447)
atgaaattaacaacacagcaaaccttgaaaaaagggtttacattaatagagctaatgattgtgattgcaattattgctattttagccactatcgcaattccctcttatcaaaattatactaaaaaagcggcggtatctgaattactgcaagcgtcagcgccttataaggctgatgtggaattatgtgtatatagtacaggtaaaccttccagttgctcgggaggaagcaatggaattgcggctgatattacaacagcaaaaggctatgtaaaatcagtgacaacaagcaacggtgcaataacagtaaaaggggatggcacattggcaaatatggaatatattttgcaagccagtggtaatgctgcaacaggtgtaacttggacaacaacttgcaaaggaacggatgcctctttatttccagcaaatttttgcggaagtgtcacacaa

SEQ ID NO: 52 - protein, 149 aa, H. influenzae
MKLTTQQTLKKGFTLIELMIVIAIIAILATIAIPSYQNYTKKAAVSELLQASAPYKADVELCVYSTGKPSSCSGGSNGIAADITTAKGYVKSVTTSNGAITVKGDGTLANMEYILQASGNAATGVTWTTTCKGTDASLFPANFCGSVTQ

SEQ ID NO: 53 - protein, 40 aa, Artificial sequence
  Synthetic Peptide
RSDYKFYEDANGTRDHKKGPSLKLLSLIKGVIVHRLEGVE

SEQ ID NO: 54 - protein, 129 aa, Artificial sequence
  Chimeric protein
GSHMTKKAAVSELLQASAPYKADVELCLVRSDYKFYEDANGTRDHKKGRHTCTGGKNGIAADITTAKGYVKSVTTSNGAITVKGDGTLANMEYILQATGNAATGVTWTTTCKGTDASLFPANFCGSVTQ

SEQ ID NO: 55 - protein, 126 aa, Artificial sequence
  Chimeric protein
GSHMTKKAAVSELLQASAPYKADVELCVYSTNETTNCTGGKNGIAADITTAKGYVKSVTTSNGAITVKGDGTLANMEYILQATGNAATGVTWTTTCLVRSDYKFYEDANGTRDHKKGRHTCGSVTQ

SEQ ID NO: 56 - protein, 138 aa, Artificial sequence
  Chimeric protein
GSHMTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQLVRSDYKFYEDANGTRDHKKGRHT

SEQ ID NO: 57 - protein, 144 aa, Artificial sequence
  Chimeric protein
GSHMLVRSDYKFYEDANGTRDHKKGRHTGPSLKLTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

(19) United States
(12) Patent Application Publication
(10) Pub. No.: US 2014/0199350 A1
(43) Pub. Date: Jul. 17, 2014

(54) CHIMERIC TOPICAL VACCINE FOR HAEMOPHILUS INFLUENZAE-INDUCED DISEASE

(71) Applicant: Research Institute at Nationwide Children's Hospital, Columbus, OH (US)

(72) Inventors: Lauren O. BAKALETZ, Hilliard, OH (US); Robert S. MUNSON, Jr., Columbus, OH (US)

(73) Assignee: Research Institute at Nationwide Children's Hospital, Columbus, OH (US)

(21) Appl. No.: 14/203,448

(22) Filed: Mar. 10, 2014

Related U.S. Application Data
(63) Continuation-in-part of application No. 13/608,500, filed on Sep. 10, 2012, now Pat. No. 8,741,304.
Application No. 13/608,500 is a continuation of application No. 12/898,287, filed on Oct. 5, 2010, now Pat. No. 8,263,363.
Application No. 12/898,287 is a division of application No. 11/994,830, filed on Jul. 9, 2008, now Pat. No. 7,811,591.
(60) Provisional application No. 60/801,835, filed on May 19, 2006.
(60) Provisional application No. 60/697,642, filed on Jul. 8, 2005.
(60) Provisional application No. 61/775,132, filed on Mar. 8, 2013.

(51) Int. Cl. A61K 39/102; A61K 9/00

(52) U.S. Cl. CPC A61K 39/102; A61K 9/0014
USPC 424/400; 424/190.1

(57) ABSTRACT

Provided are delivery systems for topically administering a chimeric protein including the NTHi twitching pilus major subunit protein (PilA) presenting a portion of the NTHi OMP P5 protein. Also provided are methods for eliciting a response by administrating the delivery systems to a subject.

This application claims priority to the following: U.S. patent application Ser. No. 13/608,500, filed Sep. 10, 2012, now allowed; U.S. patent application Ser. No. 12/898,287, filed Oct. 5, 2010, now U.S. Pat. No. 8,263,363; U.S. patent application Ser. No. 11/994,830, filed Jul. 9, 2008, now U.S. Pat. No. 7,811,591; PCT International Application No. PCT/US2006/026183, filed Jul. 5, 2006; U.S. Provisional Application No. 60/801,835 filed May 19, 2006; and U.S. Provisional Application No. 60/697,642, filed Jul. 8, 2005, and this application also claims priority to the following: U.S. Provisional Application No. 61/775,132 filed Mar. 8, 2013, each of which is incorporated by reference herein in its entirety.

### FIELD

The subject matter described herein relates to a chimeric topical vaccines for Haemophilus influenza-induced disease.

### BACKGROUND

The clinical term for middle ear infections is otitis media (OM). According to Klein, Vaccine, 19 (Suppl. 1): S2-S8, 2000, OM is the most common reason for an ill child to obtain healthcare and for a child in the United States to receive antibiotics or undergo general anesthesia. Statistics indicate that 24.5 million physician office visits were made for OM in 1990, representing a greater than 200% increase over those reported in the 1980s. While rarely associated with mortality, the morbidity associated with OM is significant. Hearing loss is a common problem associated with this disease, often affecting a child's behavior, education and development of language skills (Baldwin, Am. J. Otol., 14: 601-604, 1993; Hunter et al., Ann. Otol. Rhinol. Lamyngol. Suppl., 163: 59-61, 1994; Teele et al., J. Infect. Dis., 162: 685-694, 1990). The socioeconomic impact of OM is also great, with direct and indirect costs of diagnosing and managing OM exceeding $5 billion annually in the U.S. alone (Kaplan et al., Pediatr. Infect. Dis. J., 16: S9-11, 1997).

OM is thought to result from infectious, environmental and host genetics factors. Bacteria such as Haeinophilus influenzae, Streptococcus pneumnoniae and Moraxella catarrhalis are the most common infectious organisms in OM. Acute OM is a disease characterized by rapid onset and short duration of signs and symptoms of inflammation in the middle ear, while chronic OM refers to a condition that is defined by the relatively asymptomatic presence of fluid (or effusion) in the middle ear. However, in chronic OM, despite the absence of certain signs of acute infection (i.e., ear pain or lever), these abnormal middle ear fluids can persist for periods exceeding three months. Treatment of acute OM by antibiotic therapy is common, but multiple antibiotic-resistant bacteria have emerged in all three Genera of bacteria responsible for OM. Surgical management of chronic OM involves the insertion of tympanostomy tubes through the tympanic membrane of the ear while a child is under general anesthesia. While this procedure is commonplace (prevalence rates are ˜1 million tubes inserted per year in the U. S. Bright et al., Am. J. Public Health, 83(7): 1026-8, 1993) and is highly effective in teens of relieving painful symptoms by draining the middle ear of accumulated fluids, it is invasive and carries incumbent risks (Berman et al., Pediatrics, 93(3):353-63, 1994; Bright et al., supra.; Cimons, ASM News, 60: 527-528; Paap, Ann. Pharmacother., 30(11): 1291-7, 1996). There is thus a need for additional approaches to the management and, preferably, the prevention of OM.

OM vaccine development is most advanced for S. pneumoniae, the primary causative agent of acute OM (AOM), as evidenced by the recent approval and release of a seven-valent capsular-conjugate vaccine, PREVNAR® (Eskola and Kilpi, Pedriatr. Infect. Dis. J. 16: S72-78, 2000). While PREVNAR® has been highly efficacious for invasive pneumococcal disease, coverage for OM has been disappointing (6-8%) with reports of an increased number of OM cases due to serotypes not included in the vaccine (Black et al., Pedriatr. Infect. Dis J, 19: 187-195, 2000; Eskola et al., Pedriatr. Infect. Dis J., 19: S72-78, 2000; Eskola et al., N Engl. J. Med., 344: 403-409, 2001; Snow et al., Otol. Neurotol., 23: 1-2, 2002).

H. influenzae is a gram-negative bacterium that, as noted above, plays a role in OM. Clinical isolates of H. influenzae are classified either as serotypes “a” through “f” or as non-typeable depending on the presence or absence, respectively, of type-specific polysaccharide capsules on the bacteria. A vaccine for H. it type b has been developed. Like PREVNAR®, the type b H. influenzae vaccines target the polysaccharide capsule of this organism and thus the vaccine is comprised of capsule polysaccharide that has been conjugated to a protein carrier. Neither PREVNAR® or the type b H. influenzae vaccine have any efficacy for NTHI-induced respiratory tract diseases, including OM. Less progress has been made for a vaccine for non-typeable H. influenzae (NTHi) which causes approximately 20% of acute OM in children and predominates in chronic OM with effusion (Coleman et al., Inf and Immunity, 59(5), 1716-1722, 1991; Klein, Pedriatr. Infect. Dis J., 16, S5-8, 1997; Spinola et al., J. Infect. Dis., 154, 100-109, 1986). NTHi can also cause pneumonia, sinusitis, septicemia, endocarditis, epiglottitis, septic arthritis, meningitis, postpartum and neonatal infections, postpartum and neonatal sepsis, acute and chronic salpingitis, pericardis, cellulitis, osteomyelitis, endocarditis, cholecystitis, intraabdominal infections, urinary tract infection, mastoiditis, aortic graft infection, conjunctitivitis, Brazilian purpuric fever, occult bacteremia and exacerbation of underlying lung diseases such as chronic bronchitis, bronchietasis and cystic fibrosis. A prototype NTHi isolate is the low passage isolate 86-028NP which was recovered from a child with chronic OM. This strain has been well characterized in vitro (Bakaletz et al., Infect. Immun., 53: 331-5, 1988; Holmes et al., Microb. Pathog., 23: 157-66, 1997) as well as in chinchilla OM models (Bakaletz et al., Vaccine, 15: 955-61, 1997; Suzuki et al., Infect. Immun., 62: 1710-8, 1994; DeMaria et al., Infect. Immun., 64: 5187-92, 1996). The NTHi strain 86-026NP was deposited with the American Type Culture Collection, 10801 University Blvd., Manassas, Va. 20110, on Oct. 16, 2001 and assigned accession no. PTA-4764. A contig set from the genome of stain 86-028NP can be found at Columbus Children's Research Institute Center for Microbial Pathogenesis web site.

Adherence and colonization are acknowledged first steps in the pathogenesis of H. influenzae-induced diseases. As such, H. influenzae express multiple adhesins including hemagglutinating pili, fimbriae and non-fimbrial adhesins (Gilsdorf et al., Pediatr Res 39, 343-348, 1996; Gilsdorf., Infect. Immun., 65, 2997-3002, 1997; and St. Geme III, Cell. Microbiol., 4, 191-200, 2002). Notably, none of the adhesins described have previously been associated with a motility function. Moreover, H. influenzae do not express flagella which are also associated with motility. Twitching motility is a flagella-independent fowl of bacterial translocation over moist surfaces and occurs by extension, tethering, and then retraction of polar structures known as type IV pili (Bardy, Microbiology, 149, 295-304, 2003; Tonjum and Koomey, Gene, 192, 155-163, 1997; Wolfgang et al., EMBO J., 19, 6408-6418; Mattick, Annu. Rev. Microbiol., 56, 289-314, 2002). Type IV pili are typically 5-7 run in diameter, several micrometers in length and comprised of a single protein subunit assembled into a helical conformation with ˜5 subunits per turn (Bardy et al., Microbiology, 149, 295304, 2003; Wall and Kaiser, Mol. Microbiol., 32, 1-10, 1999). Type IV pilin subunits are usually 145-160 amino acids in length and may be glycosylated or phosphorylated. There are two classes of pilin subunits, type IVa and type IVb, which are distinguished from one another by the average length of the leader peptide and the mature subunit, which N-methylated amino acid occupies the N-terminal position of the mature protein, and the average length of the D-region (for disulfide region). Most of the respiratory pathogens express class IVa pilins, whereas the enteropathogens more typically express class IVb pilins. Type IVa pili are distinguished by the presence of a highly conserved, hydrophobic N-terminal methylated phenylalanine.

Type IV pili serve as a means of rapid translocation over and colonization of new surfaces. Thus type IV pilus expression is important to both adherence and biofilm formation by many bacteria (Mattick, Annu. Rev. Microbiol., 56, 289-314 2002; O'Toole and Kolter, Mol. Microbiol., 30, 295-304, 1998; Klausen et al., Mol. Microbiol., 50, 61-68, 2003; Jesaitis et al., J. Immunol., 171, 4329-4339, 2003), as well as virulence of Neisseria species, Moraxella bovis, Vibrio cholerae, enteropathogenic Escherichia coli and Pseudomonas aeruginosa, among others (O'Toole and Kolter, supra; Klausen et al., supra; Klausen et al., Mol. Microbiol., 48, 1511-1524, 2003; Strom and Lory, Annu. Rev. Microbiol., 47, 565-596, 1993). A biofilm is a complex organization of bacteria that are anchored to a surface via a bacterially extruded matrix, comprised of exopolysaccharide or other substances. The matrix envelopes the bacteria and protects it from the human immune system. Ehrlich et al., JAMA, 287(13), 1710-1715 (2002) describes biofilm formation by H. influenzae. It has been postulated that blocking the interaction between type IV pili and the human body can avoid or stop the bacterial infection (Meyer et al., U.S. Pat. No. 6,268,171 issued Jul. 31, 2001).

Type IV pilus expression is a complex and highly regulated bacterial function. In P. aeruginosa, the biogenesis and function of type IV pili is controlled by over forty genes (Strom and Lory, supra). To date, only a subset of the vast number of related type IV pilus genes (Tonjum and Koomey, supra; Darzins and Russell, Gene, 192, 109-115, 1997) have been found in several members of the HAP (Haemophilus, Actinobacillus and Pasteurella) family (Stevenson et al., Vet. Microbiol., 92, 121-134, 2003; Doughty et al., Vet. Microbiol., 72, 79-90, 2000; Dougherty and Smith, Microbiology, 145, 401-409 1999), but neither expression of type IV pili nor twitching motility has ever been described for any H. influenzae isolate. In fact, H. influenzae is classically described as a bacterium that does not express these structures (Friedrich et al. Appl. Environ. Microbiol., 69, 3695-3700, 2003; Fussenegger et al., Gene, 192, 125-134, 1997), despite the presence of a cryptic gene cluster within the strain Rd genome (Fleischmann et al., Science, 269, 496-512, 1995). Strain Rd is a non-encapsulated derivative of an H. influenzae serotype d organism (Zwahlen et al., Infect. Immun., 42, 708-715, 1983; Bendler and Goodgal, J. Microbiol., 70, 411-422, 1972; Risberg et al., Eur. J. Biochem., 261, 171180, 1999). Although strain Rd has some virulence properties, serotype d strains are generally considered to be commensals; they do not frequently cause disease (Daines et al., J. Med. Microbiol., 52, 277-282, 2003). It is therefore important to make the distinction between disease-causing strains of H. influenzae and strain Rd.

Fimbriae, which are surface appendages found on non-typable Haemophilus influenzae, are produced by 100% of the bacteria recovered from the middle ears and nasopharyngeal region of children with chronic otitis media. A vaccine comprised of fimbrin, a filamentous protein derived from the fimbriae of non-typable Haemophilus influenzae was previously developed and is useful in studying, preventing, or reducing the severity of otitis media. However, existing methodologies to isolate fimbrin protein from the bacterial outer membrane are tedious and time-consuming. Similarly, purification of fimbrin expressed by the fimbrin gene in other host vector, is also tedious due to the homology between the fimbrin protein and the outer membrane proteins of the host vector.

### SUMMARY

The presently described subject matter relates to chimeric proteins comprising a portion of the Type IV pilus major subunit protein (PilA) of nontypeable H. influenzae (NTHi) and a portion of NTHi OMP P5 protein (also called P5-fimbrin, fimbrin or OMP P5-homologous adhesin). In particular, the presently described subject matter provides for chimeric proteins comprising PilA modified to present the B-cell epitope of the LB1 peptide. The presently described subject matter also provides topical vaccine compositions comprising one or more chimeric proteins of the presently described subject matter and methods of eliciting an immune response via topical administration using the chimeric proteins of the presently described subject matter.

The LB1 peptide is a 40 amino acid synthetic chimeric P5-fimbrin derived peptide (SEQ ID NO: 53) that induces an immunogenic response to NTHi and is advantageous because it does not require tedious purification techniques. The LB1 peptide comprises an N-terminal 19 amino acid peptide that is a B-cell epitope (SEQ ID NO: 4). The B-cell epitope was derived from the predicted surface-exposed loop 3 of an outer membrane protein (fimbrin) of NTHi denoted as OMP P5 (also called P5-fimbrin or OMP P5-homologous adhesin). The LB1 peptide further comprises a short 5-mer linker peptide and a 16-residue T cell promiscuous eptiope. The T cell epitope was derived from a fusion protein of the measles virus. The T cell promiscuous epitope induces a very strong T cell response in individuals exposed to this epitope.

The presently described subject matter is directed to inserting a portion or fragment of the LB1 peptide into a safer and selective carrier protein that does not reduce the effectiveness of inducing a B-cell response. The portion of the LB1 peptide can be inserted into a carrier that itself also confers protection against NTHi-induced diseases. One such carrier that may induce protection against NTHi induced diseases is the protein that comprises the NTHi Type IV pilus (twitching pilus) protein, also known as PilA protein (SEQ ID NO: 2). The PilA protein is encoded by the pilA gene (SEQ ID NO: 1).

The presently described subject matter provides for chimeric proteins comprising a portion of the LB1 peptide in order to present the peptide to induce an immunogenic response. The presently described subject matter is directed to presenting a portion of the LB1 peptide that is 12 to 35 amino acids, presenting a portion of the LB1 peptide that is 15 to 30 amino acids, and presenting a portion of the LB1 peptide that is its to 19 amino acids and is a subunit of the fimbrin protein. A portion of the LB1 peptide can be the N-terminal amino acid sequence RSDYKFYEDANGTRDHKKG (SEQ ID NO: 4).

The presently described subject matter provides for chimeric protein wherein the PilA protein is modified to present a 24 amino acid peptide. The 24 amino acid peptide may comprise the B-cell epitope of the LB1 peptide modified as set out in the amino acid sequence of SEQ ID NO: 5 (LVRSDYKFYEDANGTRDHKKGRHT) in which a leucine and valine are added to the N terminus of the B-cell epitope of LB1 and an arginine, histidine and threonine are at the C terminus of the B-cell epitope of LB1. These modifications to the B-cell epitope are contemplated to assist in protein folding and/or antigen presentation. The presently described subject matter is further directed to any modifications to the B-cell epitope of LB1 that will assist in protein folding and/or antigen presentation.

The amino acid sequence of the surface exposed loop 3 of NTHi OMP P5 can vary between NTHi strains. The presently described subject matter is directed to chimeric proteins comprising a portion of the PilA protein modified to present the B cell epitope of any variant amino acid sequence of loop 3 of the NTHi OMP P5. In particular, the presently described subject matter provides for chimeric proteins wherein the PilA protein is modified to present one of the following variant NTHi OMP P5 amino acids sequences: RSDYKLYNKNSSSNSTLKNLGE (SEQ ID NO: 6), RSDYKLYNKNSSTLKDLGE (SEQ ID NO: 7) and RSDYKFYDNKRID (SEQ ID NO: 8). The variant peptides also may be presented with a leucine and valine added to the N terminus and an arginine, histidine and threonine added to the C terminus or any other modification to assist in protein folding and/or antigen presentation.

The chimeric proteins of the presently described subject matter can comprise the modified PilA amino acids wherein the native PilA amino acids have been substituted with a portion of the LB1 peptide. In addition, the chimeric proteins of the presently described subject matter can comprise a modified PilA amino acid sequence wherein a portion of the LB1 peptide is inserted within and in addition to the native PilA amino acids. The chimeric proteins of the presently described subject matter have the ability to induce the formation of antibodies directed against two proteins and therefore are more effective and more specific topical vaccines.

The chimeric proteins can comprise the mature amino acid sequence (residues 13-149) of the NTHi Pi1A protein (SEQ ID NO: 2) wherein a portion of the LB1 peptide is inserted between the cysteine residues at positions 62 and 72 of SEQ ID NO: 2 and may substitute the native amino acids, such as the chimeric protein having the amino acid sequence of SEQ ID NO: 54. This chimeric protein comprises residues 40-149 of SEQ ID NO: 2 and has the B-cell epitope of LB1 (SEQ ID NO: 5) inserted between residues 62 and 72 of SEQ ID NO: 2. The portion of the LB1 peptide is inserted between the cysteine residues at positions 131 and 144 of SEQ ID NO: 2 and may substitute the native amino acids such as the protein having the amino acid sequence of SEQ ID NO: 55. This chimeric protein can comprise residues 40-149 of SEQ ID NO: 2 and has the B-cell epitope of LB1 (SEQ ID NO: 5) inserted between residues 131 and 144 of SEQ ID NO: 2.

The chimeric proteins can comprise the mature amino acid sequence (residues 13-149) of the NTHi PilA protein (SEQ ID NO: 2) where the portion of the LB1 peptide is inserted at the C-terminus of the PilA protein. For example, the chimeric protein of SEQ ID NO: 56 can comprise residues 40-149 of SEQ ID NO: 2 and the B-cell epitope of LB1 (SEQ ID NO: 5) is inserted following residue 149 of SEQ ID NO: 2.

The chimeric proteins can comprise the mature amino acid sequence (residues 13-149) of NTHi PilA protein (SEQ ID NO: 2) where the portion of the LB1 peptide is inserted at the N-terminus of the PilA protein. For example, the chimeric protein of SEQ ED NO: 57 can comprise residues 40-149 of SEQ ID NO: 2 and the B-cell epitope of LB1 (SEQ ID NO: 5) is inserted before residue 40 of SEQ ID NO: 2.

The presently described subject matter provides for chimeric proteins that can comprise a portion of the NTHi PilA protein and one or more of the LB1 peptides described herein. The chimeric proteins of the presently described subject matter include those which present the same LB1 peptide more than once within a portion of the NTHi PilA protein and those which present two or more different LB1 peptides within a portion of the NTHi PilA protein.

The presently described subject matter further provides for chimeric proteins that can comprise a portion of the NTHi PilA protein and any antigenic protein that will elicit an immune response upon topical administration or application.

The presently described subject matter is directed to a topical delivery system, comprising a topical immunogenic composition comprising at least one active agent comprising a chimeric protein comprising residues 40-149 of SEQ ID NO: 2 and SEQ ID NO: 4, SEQ ID NO: 4 being inserted before residue 40 of SEQ ID NO: 2; and a flexible substrate in communication with the topical immunogenic composition.

The presently described flexible substrate can comprise a skin-contacting surface. The flexible substrate can conform to an area of skin to which it is applied. The flexible substrate can comprise at least one layer comprising a flexible material and having a skin-contacting surface. The topical immunogenic composition can be provided on the skin-contacting surface of the flexible substrate. The topical immunogenic composition can be dispersed throughout the flexible substrate or throughout at least one layer, having a skin-contacting surface, of the flexible substrate.

The presently described subject matter is directed to a topical delivery system where the topical immunogenic composition further comprises at least one pharmaceutically acceptable topical carrier.

The presently described subject matter is directed to a topical delivery system where the at least one pharmaceutically acceptable topical carrier can comprise any known pharmaceutically acceptable topical carrier.

The presently described subject matter is directed to a topical delivery system, wherein the active agent comprises the amino acid sequence of SEQ ID NO: 57.

The presently described subject matter is directed to a topical delivery system where the topical immunogenic composition comprises a formulation selected from a gel, an ointment, a solution, an emulsion, a dispersion, a suspension, and a cream.

The presently described subject matter is directed to a topical delivery system, comprising a topical immunogenic composition comprising at least one active agent comprising a chimeric protein comprising residues 40-149 of SEQ ID NO: 2 and SEQ ID NO: 4, SEQ ID NO: 4 being inserted before residue 40 of SEQ ID NO: 2.

The presently described subject matter is directed to a method for eliciting an immune response to NTHi bacteria, in a patient, comprising topically administering to the patient at risk of NTHi bacterial infection, a topical delivery system comprising a topical immunogenic composition comprising at least one active agent, the at least one active agent comprising an immunogenic dose of a chimeric protein comprising an immunogenic PilA peptide selected from the group consisting of the amino acid sequence of SEQ ID NO: 6, SEQ ID NO: 7, SEQ ID NO: 8, and residues 40 to 149 of SEQ ID NO: 2, and a portion of the LB1 peptide selected from the group consisting of the amino acid sequence of SEQ ID NO: 4 and SEQ ID NO: 5, wherein the LB1 peptide is inserted before the immunogenic PilA peptide.

The presently described subject matter is directed to a method wherein the PilA peptide comprises residues 40-149 of SEQ ID NO: 2 and wherein the LB1 peptide comprises SEQ ID NO: 4.

The presently described subject matter is directed to a method wherein the chimeric protein comprises the amino acid sequence of SEQ ID NO: 57.

The presently described subject matter is directed to a method wherein administering comprises applying the topical delivery system at least once.

The presently described subject matter is directed to a method wherein administering comprises applying the topical delivery system once a week, at weekly intervals, for a pre-determined period of time.

The presently described subject matter is directed to a method wherein the predetermined period of time is from 2 weeks to 10 weeks.

The presently described subject matter is directed to a method wherein the predetermined period of time is 2 weeks.

The presently described subject matter is directed to a method wherein the topical delivery system remains on the patient for a period of at least 24 hours.

The presently described subject matter is directed to a method wherein administering comprises applying the topical delivery system to an area of skin of the patient.

The presently described subject matter is directed to a method wherein the topical delivery system further comprises a flexible substrate.

The presently described subject matter is directed to a method wherein the NTHi infection is in the middle ear, nasopharynx or lower airway.

The presently described subject matter is directed to a method wherein the NTHi bacterial infection involves a pathological condition selected from the group consisting of otitis media, pneumonia, sinusitis, septicemia, endocarditis, epiglottitis, septic arthritis, meningitis, sepsis, salpingitis, epiglottis, pericardis, cellulitis, osteomyelitis, endocarditis, cholecystitis, intraabdominal infections, urinary tract infection, mastoiditis, aortic graft infection, conjunctitivitis, Brazilian purpuric fever, occult bacteremia, chronic obstructive pulmonary disease, chronic bronchitis, bronchietasis, and cystic fibrosis.

The presently described subject matter is directed to a method for treating a NTHi bacterial infection in a patient, comprising topically administering to the patient, a topical delivery system comprising a topical immunogenic composition comprising at least one active agent, the at least one active agent comprising an immunogenic dose of a chimeric protein comprising an immunogenic PilA peptide selected from the group consisting of the amino acid sequence of SEQ ID NO: 6, SEQ ID NO: 7, SEQ ID NO: 8, and residues 40 to 149 of SEQ ID NO: 2, and a portion of the LB1 peptide selected from the group consisting of the amino acid sequence of SEQ ID NO: 4 and SEQ ID NO: 5, wherein the LB1 peptide is inserted before the immunogenic PilA peptide.

The presently described subject matter is directed to a method wherein the patient suffers from a pathological condition selected from the group consisting of otitis media, pneumonia, sinusitis, septicemia, endocarditis, epiglottitis, septic arthritis, meningitis, sepsis, salpingitis, epiglottis, pericardis, cellulitis, osteomyelitis, endocarditis, cholecystitis, intraabdominal infections, urinary tract infection, mastoiditis, aortic graft infection, conjunctitivitis, Brazilian purpuric fever, occult bacteremia, chronic obstructive pulmonary disease, chronic bronchitis, bronchietasis, and cystic fibrosis.

### BRIEF DESCRIPTION OF THE DRAWING

FIG. 1 provides the timeline of the immunization regimen, viral inoculation, bacterial challenge, and OM disease assessment period for the efficacy experiments described in Example 5.

### DETAILED DESCRIPTION

### The NTHi Type IV Pilus (PilA) Polynucleotides and Polypeptides of the Presently Described Subject Matter

The chimeric proteins of the presently described subject matter may comprise the full length or a portion of the major subunit of the NTHi Type IV Pilus which is encoded by the gene pilA. The PilA protein of the NTHi isolate 86-028NP is encoded by the nucleic acid sequence set out as SEQ ID NO: 2, which is described in U.S. patent application Ser. No. 11/019,005, incorporated by reference herein in its entirety. Also provided are polynucleotides encoding PilA polypeptides from NTHi clinical isolates 1728MEE, 1729MEE, 3224A, 10548MEE, 1060MEE, 1885MEE, 1714MEE, 1236MEE, 1128MEE and 214NP. The amino acid sequences of these Pi1A polypeptides are set out in SEQ ID NOS: 34, 36, 38, 40, 42, 44, 46, 48, 50 and 52 respectively. The possibility of alternative codon usage is specifically contemplated in polynucleotides encoding the polypeptides. In one embodiment, the polypeptides are respectively encoded by the nucleotide sequences set out in SEQ ID NOS: 33, 35, 37, 39, 41, 43, 45, 47, 49 and 51.

The presently described subject matter provides for polynucleotides that hybridize under stringent conditions to (a) the complement of the nucleotide sequences set out in SEQ ID NOS: 1, 33, 35, 37, 39, 41, 43, 45, 47, 49 and 51; (b) a polynucleotide which is an allelic variant of any polynucleotides recited above; (c) a polynucleotide which encodes a species homolog of any of the proteins recited above; or (d) a polynucleotide that encodes a polypeptide comprising a specific domain or truncation of the polypeptides of the presently described subject matter. PilA polynucleotides from other non-typeable H. influenzae strains and from H. influenzae strains a, b, c, e and f are specifically contemplated. These polynucleotides can be identified and isolated by techniques standard in the art such as hybridization and polymerase chain reaction using part or all of the polynucleotides of SEQ ID NOS: 1, 33, 35, 37, 39, 41, 43, 45, 47, 49 and 51 as probes or primers, respectively.

The polynucleotides of the presently described subject matter also include nucleotide sequences that are substantially equivalent to the polynucleotides recited above. Polynucleotides according to the presently described subject matter can have, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, or 99% sequence identity to the NTHi polynucleotides recited above.

Included within the scope of the nucleic acid sequences of the presently described subject matter are nucleic acid sequence fragments that hybridize under stringent conditions to the NTHi nucleotide sequences of SEQ ID NOS: 1, 33, 35, 37, 39, 41, 43, 45, 47, 49 and 51, or complements thereof, which fragment is greater than about 5 nucleotides, 7 nucleotides, greater than 7 nucleotides, greater than 9 nucleotides, and greater than 17 nucleotides. Fragments of, e.g., 15, 17, or 20 nucleotides or more that are selective for (i.e., specifically hybridize to any one of the PilA polynucleotides of the presently described subject matter) are suitable. These nucleic acid sequence fragments capable of specifically hybridizing to an NTHi PilA polynucleotide of the presently described subject matter can be used as probes to detect NTHi PilA polynucleotides of the presently described subject matter and/or can differentiate NTHi PilA polynucleotides of the presently described subject matter from other bacterial genes, and are preferably based on unique nucleotide sequences.

The term “stringent” is used herein to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2^{nd} Ed., Cold Spring Harbor Laboratory, (Cold Spring Harbor, N.Y. 1989).

More stringent conditions (such as higher temperature, lower ionic strength, higher formamide, or other denaturing agent) may also be used; however, the rate of hybridization will be affected. In instances where hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples include 0.1% bovine serum albumin, 0.1% polyvinyl-pyrrolidone, 0.1% sodium pyrophosphate, 0.1% sodium dodecylsulfate, NaDodSO4, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

As noted above, suitable polynucleotides are not limited to the specific PilA polynucleotides of SEQ ID NOS: 1, 33, 35, 37, 39, 41, 43, 45, 47, 49 and 51, but also include, for example, allelic and species variations thereof. Allelic and species variations can be routinely determined by comparing the sequence provided in SEQ ID NOS: 1, 33, 35, 37, 39, 41, 43, 45, 47, 49 and 51, preferably the open reading frames therein, a representative fragment thereof, or a nucleotide sequence at least 90% identical, preferably 95% identical, to the open reading frames within SEQ ID NOS: 1, 33, 35, 37, 39, 41, 43, 45, 47, 49 and 51 with a sequence from another isolate of the same species or another species. Computer program methods to determine identity and similarity between two sequences include, but are not limited to, the GCG program package, including GAP (Devereux et al., Nucl. Acid. Res., 12: 387, 1984; Genetics Computer Group, University of Wisconsin, Madison, Wis.), BLASTP, BLASTN, and FASTA (Altschul et al., J. Mol. Biol., 215: 403-410, 1990). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith-Waterman algorithm may also be used to determine identity.

Polynucleotides of the presently described subject matter may be isolated from natural sources or may be synthesized by standard chemical techniques, e.g., the phosphotriester method described in Matteucci et al., J Am Chem. Soc., 103: 3185 (1981).

The presently described subject matter provides for chimeric proteins comprising a portion of NTHi PilA protein. The polypeptides can comprise the NTHi 86-028NP amino acid sequences respectively set out in SEQ ID NO: 2. Polypeptides of the presently described subject matter may also include PilA polypeptides set out in SEQ ID NOS: 34, 36, 38, 40, 42, 44, 46, 48, 50 and 52. The PilA polypeptides of the presently described subject matter can be those of other non-typeable H. influenzae strains and from H. influenzae strains a, b, c, e and f.

Polypeptides of the presently described subject matter specifically include peptide fragments (i.e., peptides) or fragments of the PilA polypeptide that retain one or more biological or immunogenic properties of a full length polypeptide of the presently described subject matter. In one embodiment, PilA peptide fragments provided by the presently described subject matter are designated TfpQ2, TfpQ3, TfpQ4 and OLP3 and respectively comprise amino acids 35 through 68 of SEQ ID NO: 2, amino acids 69 through 102 of SEQ ID NO: 2, amino acids 103 through 137 of SEQ ID NO: 2, and amino acids 21 through 35 of SEQ ID NO: 2. Another PilA peptide fragment provided by the presently described subject matter comprises amino acids 40 through 149 of SEQ ID NO: 2.

The presently described subject matter also provides for chimeric proteins comprising a portion of a PilA polypeptide with one or more conservative amino acid substitutions that do not affect the biological and/or immunogenic activity of the PilA polypeptide. Alternatively, the PilA polypeptides of the presently described subject matter can have conservative amino acids substitutions which may or may not alter biological activity. The term “conservative amino acid substitution” refers to a substitution of a native amino acid residue with a normative residue, including naturally occurring and nonnaturally occurring amino acids, such that there is little or no effect on the polarity or charge of the amino acid residue at that position. For example, a conservative substitution results from the replacement of a non-polar residue in a polypeptide with any other non-polar residue. Further, any native residue in the polypeptide may also be substituted with alanine, according to the methods of “alanine scanning mutagenesis.” Naturally occurring amino acids are characterized based on their side chains as follows: basic: arginine, lysine, histidine; acidic: glutamic acid, aspartic acid; uncharged polar: glutamine, asparagine, serine, threonine, tyrosine; and non-polar: phenylalanine, tryptophan, cysteine, glycine, alanine, valine, proline, methionine, leucine, norleucine, isoleucine. General rules for amino acid substitutions are set forth in Table 1 below.

TABLE 1

Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asn |
| Gly | Pro, Ala | Ala |
| His | Asn, Gln, Lys, Arg | Arg |
| Ile | Leu, Val, Met, Ala, Phe, | Leu |
| Leu | Norleucine, Ile, Val, Met, | Leu |
| Lys | Arg, 1,4 Diaminobutyric | Arg |
| Met | Leu, Phe, Ile | Leu |
| Phe | Leu, Val, Ile, Ala, Tyr | Arg |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, | Leu |

The presently described subject matter also provides for chimeric proteins comprising a portion of a variants of the NTHi PilA polypeptides of the presently described subject matter (e.g., a polypeptide exhibiting at least about 65%, at least about 70%, at least about 75%, at least about 80%, at least about 85%, 86%, 87%, 88%, 89%, at least about 90%, 91%, 92%, 93%, 94%, at least about 95%, 96%, 97%, at least about 98%, or at least about 99% amino acid identity to a polypeptide of SEQ ID NOS: 2, 34, 36, 38, 40, 42, 44, 46, 48, 50 and 52) that retain biological and/or immunogenic activity.

The presently described subject matter is directed to PilA polynucleotides of the presently described subject matter that may be inserted in a vector for amplification or expression. For expression, the polynucleotides are operatively linked to appropriate expression control sequences such as promoter and polyadenylation signal sequences. Further provided are host cells comprising polynucleotides of the presently described subject matter. Exemplary prokaryotic host cells can include bacteria such as E. coli, Bacillus, Streptomyces, Pseudomonas, Salmonella, and Serratia. The presently described subject matter is directed to methods of producing the polypeptides of the presently described subject matter by growing the host cells and isolating polypeptide from the host cells or growth medium. Alternatively, polypeptides of the presently described subject matter can be prepared by chemical synthesis using standard means. Particularly convenient are solid phase techniques (see, e.g., Erikson et al., The Proteins (1976) v. 2, Academic Press, New York, p. 255). Automated solid phase synthesizers are commercially available. In addition, modifications in the sequence are easily made by substitution, addition or omission of appropriate residues. For example, a cysteine residue may be added at the carboxy terminus to provide a sulfhydryl group for convenient linkage to a carrier protein, or spacer elements, such as an additional glycine residue, may be incorporated into the sequence between the linking amino acid at the C-terminus and the remainder of the peptide.

The term “isolated” refers to a substance removed from, and essentially free of, the other components of the environment in which it naturally exists. For example, a polypeptide is separated from other cellular proteins or a DNA is separated from other DNA flanking it in a genome in which it naturally occurs.

Recombinant PilA protein (rPilA) may be generated to serve as a more readily renewable product. To do this, the published protocol of Keizer et al. (J. Biol. Chem., 276: 24186-14193, 2001), who studied a pilin which also had four Cys residues as it will be critical that rPilA similarly be properly folded so as to possess functional qualities of the native pilin subunit, is utilized. Briefly, a truncated pilin is engineered wherein the first 28 residues are removed from the N-terminus to prevent aggregation, and this truncated pilin will be further engineered to be transported to the periplasm by means of the incorporation of an OmpA leader sequence in the construct. Using this strategy Keizer et al. generated a recombinant soluble monomeric P. aeruginosa pilin protein that was able to bind to its receptor (asialo GM1) in in vitro assays and decrease morbidity and mortality in mice when the peptide was delivered 15 minutes prior to heterologous challenge. This soluble, monomeric, truncated form of NTHi PilA will be useful in the studies described herein.

The presently described subject matter also provides for synthetic chimeric proteins. The chimeric proteins may be synthesize, purified and sequenced using standard techniques. For example, the chimeric proteins may be assembled semi-manually by stepwise Fmoc-tert-butyl solid-phase synthesis and purified by HPLC. The composition and amino acid sequence of recombinant and synthetic chimeric proteins may be confirmed by amino acid analysis and/or mass spectral analysis.

### Antibodies

The presently described subject matter provides antibodies which bind to antigenic epitopes of the chimeric proteins of the presently described subject matter. The antibodies may be polyclonal antibodies, monoclonal antibodies, antibody fragments which retain their ability to bind their unique epitope (e.g., Fv, Fab and F(ab)2 fragments), single chain antibodies and human or humanized antibodies. Antibodies may be generated by techniques standard in the art using chimeric protein(s) of the presently described subject matter or host cells expressing chimeric protein(s) of the presently described subject matter as antigens.

The presently described subject matter provides for antibodies specific for the chimeric proteins of the presently described subject matter and fragments thereof, which exhibit the ability to kill both H. influenzae bacteria and to protect humans from infection. The presently described subject matter also provides for antibodies specific for the chimeric proteins of the presently described subject matter which reduce the virulence, inhibit adherence, inhibit biofilm formation, inhibit twitching motility, inhibit cell division, and/or inhibit penetration into the epithelium of H. influenzae bacteria and/or enhance phagocytosis of the H. influenzae bacteria.

In vitro complement mediated bactericidal assay systems (Musher et al., Infect. In nun. 39: 297-304, 1983; Anderson et al., J. Clin. Invest. 51: 31-38, 1972) may be used to measure the bactericidal activity of anti-chimeric proteins antibodies.

It is also possible to confer short-term protection to a host by passive immunotherapy via the administration of pre-formed antibody against a chimeric protein of the presently described subject matter. Thus, antibodies of the presently described subject matter may be used in passive immunotherapy. Human immunoglobulin is preferred in human medicine because a heterologous immunoglobulin may provoke an immune response to its foreign immunogenic components. Such passive immunization can, for example, be used on an emergency basis for immediate protection of unimmunized individuals subject to special risks.

Antibodies of the presently described subject matter may be used in the production of anti-idiotypic antibody, which in turn can be used as an antigen to stimulate an immune response against the chimeric protein epitopes or H. influenzae epitopes.

### Methods for Eliciting an Immune Response and Compositions Therefor

The presently described subject matter is directed to methods of eliciting in an individual an immune response to H. influenzae. For example, the methods can elicit an immune response to the chimeric proteins of the presently described subject matter. These methods can elicit one or more immune responses, including but not limited to, immune responses which inhibit bacterial replication, immune responses which block H. influenzae adherence to cells, immune responses which prevent H. influenzae twitching, immune responses that kill H. influenzae bacteria and immune responses which prevent biofilm formation. The methods can comprise a step of topically administering an immunogenic dose of a composition comprising one or more chimeric proteins of the presently described subject matter. The methods can comprise topically administering an immunogenic dose of a composition comprising a cell expressing one or more chimeric proteins of the presently described subject matter. Further, the methods can comprise topically administering an immunogenic dose of a composition comprising one or more polynucleotides encoding one or more chimeric proteins of the presently described subject matter. The polynucleotide may be a naked polynucleotide not associated with any other nucleic acid or may be in a vector such as a plasmid or viral vector (e.g., adeno-associated virus vector or adenovirus vector). The methods may be used in combination in a single individual. The methods may be used prior or subsequent to H. influenzae infection of an individual. The methods and compositions of the presently described subject matter may be used to topically treat or prevent any pathological condition involving H. influenzae (typeable and nontypeable strains) such as OM, pneumonia, sinusitis, septicemia, endocarditis, epiglottitis, septic arthritis, meningitis, postpartum and neonatal infections, postpartum and neonatal sepsis, acute and chromic salpingitis, epiglottis, pericardis, cellulitis, osteomyelitis, endocarditis, cholecystitis, intraabdominal infections, urinary tract infection, mastoiditis, aortic graft infection, conjunctitivitis, Brazilian purpuric fever, occult bacteremia, and chronic obstructive pulmonary disease and exacerbation of underlying lung diseases such as chronic bronchitis, bronchietasis, and cystic fibrosis.

In methods of the presently described subject matter, a composition of the presently described subject matter can be topically administered as a priming dose followed by one or more topical booster doses. Proteins or polypeptides that beneficially enhance the immune response such as cytokines (e.g., IL-2, IL-12, GM-CSF), cytokine-inducing molecules (e.g. Leaf) or co-stimulatory molecules can be topically co-administered.

An “immunogenic dose” of a composition of the presently described subject matter is one that generates, after topical administration, a detectable humoral (antibody) and/or cellular (T cell) immune response in comparison to the immune response detectable before topical administration or in comparison to a standard immune response before topical administration. The immune response resulting from the presently described methods may be protective and/or therapeutic. The antibody and/or T cell immune response can protect the individual from H. influenzae infection, for example, infection of the middle ear and/or the nasopharynx or lower airway. In this use, the precise dose depends on the patient's state of health and weight, topical administration, the nature of the formulation, etc., but generally ranges from about 1.0 μg to about 5000 μg per 70 kilogram patient, more commonly from about 10 to about 500 μg per 70 kg of body weight.

Humoral immune response may be measured by many well known methods, such as Single Radial Immunodiffussion Assay (SRID), Enzyme Immunoassay (ETA) and Hemagglutination Inhibition Assay (HAI). In particular, SRID utilizes a layer of a gel, such as agarose, containing the immunogen being tested. A well is cut in the gel and the serum being tested is placed in the well. Diffusion of the antibody out into the gel leads to the formation of a precipitation ring whose area is proportional to the concentration of the antibody in the serum being tested. ETA, also known as ELISA (Enzyme Linked Immunoassay), is used to determine total antibodies in the sample. The immunogen is adsorbed to the surface of a microtiter plate. The test serum is exposed to the plate followed by an enzyme linked immunoglobulin, such as IgG. The enzyme activity adherent to the plate is quantified by any convenient means such as spectrophotometry and is proportional to the concentration of antibody directed against the immunogen present in the test sample. HAI utilizes the capability of an immunogen such as viral proteins to agglutinate chicken red blood cells (or the like). The assay detects neutralizing antibodies, i.e., those antibodies able to inhibit hemagglutination. Dilutions of the test serum are incubated with a standard concentration of immunogen, followed by the addition of the red blood cells. The presence of neutralizing antibodies will inhibit the agglutination of the red blood cells by the immunogen. Tests to measure cellular immune response include determination of delayed-type hypersensitivity or measuring the proliferative response of lymphocytes to target immunogen.

The presently described subject matter correspondingly provides topical compositions suitable for eliciting an immune response to chimeric proteins of the presently described subject matter. As noted above, the topical compositions can comprise one or more chimeric proteins, cells expressing one or more chimeric proteins, or one or more polynucleotides encoding one or more chimeric proteins. The topical compositions may also comprise one or more other ingredients such as pharmaceutically acceptable topical carriers and adjuvants.

In topical compositions of the presently described subject matter, a chimeric protein may be fused to another protein when produced by recombinant methods. For example, the other protein may not, by itself, elicit antibodies, but it may stabilize the first protein and forms a fusion protein retaining immunogenic activity. Further, the fusion protein can comprise another protein that is immunogenic, such as Glutathione-S-transferase (GST) or beta-galactosidase, relatively large co-proteins which solubilize the fusion protein and facilitate production and purification thereof. The other protein may act as an adjuvant in the sense of providing a generalized stimulation of the immune system. The other protein may be fused to either the amino or carboxy terminus of the chimeric proteins of the presently described subject matter.

In other topical compositions of the presently described subject matter, chimeric proteins may be otherwise linked to carrier substances. Any method of creating such linkages known in the art may be used. Linkages can be formed with hetero-bifunctional agents that generate a disulfide link at one functional group end and a peptide link at the other, such as a disulfide amide forming agent, e.g., N-succidimidyl-3-(2-pyridyldithio) proprionate (SPDP) (see, e.g., Jansen et al., Immun. Rev. 62:185, 1982) and bifunctional coupling agents that form a thioether rather than a disulfide linkage such as reactive esters of 6-maleimidocaproic acid, 2-bromoacetic acid, 2-iodoacetic acid, 4-(N-maleimido-methyl)cyclohexane-1-carboxylic acid and the like, and coupling agent which activate carboxyl groups by combining them with succinimide or 1-hydroxy-2-nitro-4-sulfonic acid, for sodium salt such as succinimmidyl 4-(N-maleimido-methyl)cyclohexane-1-carobxylate (SMCC).

The present chimeric proteins may be formulated as neutral or salt forms. Pharmaceutically acceptable salts, include the acid addition salts (formed with the free amino groups of the peptide) and which are formed with inorganic acids such as, e.g., hydrochloric or phosphoric acids, or such organic acids as acetic, oxalic, tartaric, mandelic. Salts formed with the free carboxyl groups may also be derived from inorganic bases such as, e.g., sodium, potassium, ammonium, calcium, or ferric hydroxides, and such organic bases as isopropylamine, trimethylamine, 2-ethylamino ethanol, histidine, and procaine.

Topical compositions of the presently described subject matter may further comprise one or more adjuvants, including for example, known adjuvants. Known adjuvants include, for example, emulsions such as Freund's Adjuvants and other oil emulsions, Bordetella pertussis, MF59, purified saponin from Quillaja saponaria (QS21), aluminum salts such as hydroxide, phosphate and alum, calcium phosphate, (and other metal salts), gels such as aluminum hydroxide salts, mycobacterial products including muramyl dipeptides, solid materials, particles such as liposomes and virosomes. Examples of natural and bacterial products known to be used as adjuvants include monophosphoryl lipid A (MPL), RC-529 (synthetic MPL-like acylated monosaccharide), OM-174 which is a lipid A derivative from E. coli, holotoxins such as cholera toxin (CT) or one of its derivatives, pertussis toxin (PT) and heat-labile toxin (LT) of E. coli or one of its derivatives, and CpG oligonucleotides. Adjuvant activity can be affected by a number of factors, such as carrier effect, depot formation, altered lymphocyte recirculation, stimulation of T-25 lymphocytes, direct stimulation of B-lymphocytes and stimulation of macrophages.

Suitable excipients include, e.g., water, saline, dextrose, glycerol, or the like and combinations thereof. In addition, if desired, the topical vaccine may contain minor amounts of auxiliary substances such as wetting or emulsifying agents, pH buffering agents, or adjuvants, which can enhance the effectiveness of the topical vaccine. The topical vaccines are administered topically.

Topical compositions may also be administered utilizing jet injectors, microneedles, electroporation, sonoporation, microencapsulation, polymers or liposomes, transmucosal routes and intranasal routes using nebulizers, aerosols and nasal sprays. Microencapsulation using natural or synthetic polymers such as starch, alginate and chitosan, D-poly L-lactate (PLA), D-poly DL-lactic-coglycolic microspheres, polycaprolactones, polyorthoesters, polyanhydrides and polyphosphazenes polyphosphatazanes are useful for both transdermal and transmucosal administration. Polymeric complexes comprising synthetic poly-omithate, poly-lysine and poly-arginine or amphipathic peptides are useful for transdermal delivery systems. In addition, due to their amphipathic nature, liposomes are contemplated for transdermal, transmucosal and intranasal vaccine delivery systems. Common lipids used for topical vaccine delivery include N-(1)2,3-(dioleyl-dihydroxypropyl)-N,N,N-trimethylammonium-methyl sulfate (DOTAP), dioleyloxy-propyl trimethylammonium chloride DOTMA, dimystyloxpropyl-3-dimethyl-hydroxyethyl ammonium (DMRIE), dimethyldioctadecyl ammonium bromide (DDAB) and 9N(N′,N-dimethylaminoethane) carbamoyl) cholesterol (DC-Chol). The combination of helper lipids and liposomes can enhance up-take of the liposomes through the skin. These helper lipids include dioleoyl phosphatidylethanolamine (DOPE), dilauroylphosphatidylethanolamine (DLPE), dimyristoyl phosphatidylethanolamine (DMPE), dipalmitoylphosphatidylethanolamine (DPPE). In addition, triterpenoid glycosides or saponins derived from the Chilean soap tree bark (Quillaja saponaria) and chitosan (deacetylated chitan) have been contemplated as useful adjuvants for intranasal and transmucosal topical vaccine delivery.

Topical formulations may be presented in unit-dose or multi-dose containers.

Methods of Inhibiting H. influenzae

Alternatively, the presently described subject matter includes methods of inhibiting H. influenzae type IV pili function in an individual. The methods comprise topically administering to the individual, for example, one or more antibodies of the presently described subject matter and/or one or more chimeric proteins of the presently described subject matter; in an amount that inhibits function of the pili. In vitro assays may be used to demonstrate the ability to inhibit pili function. These methods can include, for example, methods using inhibitors of adherence mediated via type IV pili, inhibitors that disrupt existing biofilms mediated by type IV pili, and inhibitors of twitching.

Inhibition is contemplated for any pathological condition involving H. influenzae, for example, OM, pneumonia, sinusitis, septicemia, endocarditis, epiglottitis, septic arthritis, meningitis, postpartum and neonatal infections, postpartum and neonatal sepsis, acute and chromic salpingitis, epiglottis, pericardis, cellulitis, osteomyelitis, endocarditis, cholecystitis, intraabdominal infections, urinary tract infection, mastoiditis, aortic graft infection, conjunctitivitis, Brazilian purpuric fever, occult bacteremia, chronic obstructive pulmonary disease and exacerbation of underlying lung diseases such as chronic bronchitis, bronchietasis and cystic fibrosis.

Topical compositions comprising inhibitors of H. influenzae type IV pili function are provided. The topical compositions may consist of one of the foregoing active ingredients alone, may comprise combinations of the foregoing active ingredients or may comprise additional active ingredients used to treat bacterial infections. As discussed above, the topical compositions may comprise one or more additional ingredients such as pharmaceutically effective topical carriers. Also as discussed above, dosage and frequency of the topical administration of the topical compositions are determined by standard techniques and depend, for example, on the weight and age of the individual, area of topical administration, and the severity of symptoms. Topical administration of the pharmaceutical compositions may be, for example, by application to an area of skin, oral, buccal, nasal, rectal, intranasal, or vaginal.

### Animal Model

Methods of the presently described subject matter may be demonstrated in a chinchilla model widely accepted as an experimental model for OM. In particular, a chinchilla model of NTHi-induced OM has been well characterized (Bakaletz et al., J. Infect. Dis., 168: 865-872, 1993; Bakaletz and Holmes, Clin. Diagn. Lab. Immunol., 4: 223-225, 1997; Suzuki and Bakaletz, Infect. Immun., 62: 1710-1718, 1994; Mason et al., Infect. Immun., 71:3454-3462, 2003), and has been used to determine the protective efficacy of several NTHi outer membrane proteins, combinations of outer membrane proteins, chimeric synthetic peptide vaccine components, and adjuvant formulations against OM (Bakaletz et al., Vaccine, 15: 955-961, 1997; Bakaletz et al., Infect. Immun., 67: 2746-2762, 1999; Kennedy et al., Infect. Immun., 68: 2756-2765, 2000; Kyd et al., Infect. Immun., 66:2272-2278, 2003; Novotny and Bakaletz, J. Immunol., 171, 1978-1983, 2003).

In the model, adenovirus predisposes chinchillas to H. influenzae-induced OM media, which allowed for the establishment of relevant cell, tissue and organ culture systems for the biological assessment of NTHi (Bakaletz et al., J. Infect. Dis., 168: 865-72, 1993; Suzuki et al., Infect. Immunity 62: 1710-8, 1994). Adenovirus infection alone has been used to assess the transudation of induced serum antibodies into the tympanum (Bakaletz et al., Clin. Diagnostic Lab Immunol., 4(2): 223-5, 1997) and has been used as a co-pathogen with NTHi, to determine the protective efficacy of several active and passive immunization regimens targeting various NTHi outer membrane proteins, combinations of OMPs, chimeric synthetic peptide vaccine components, and adjuvant formulations as vaccinogens against otitis media (Bakaletz et al., Infect Immunity, 67(6): 2746-62, 1999; Kennedy et al., Infect. Immun., 68(5): 2756-65, 2000; Novotny et al., Infect Immunity 68(4): 2119-28, 2000; Poolman et al., Vaccine 19 (Suppl. 1): S108-15, 2000).

### EXAMPLES

The following examples illustrate the presently described subject matter where Example 1 describes recombinant production of chimeric proteins of the presently described subject matter, Example 2 describes assays to test the immunogenicity of the chimeric proteins of the presently described subject matter, Example 3 describes assays for evaluating passive immunization, Example 4 describes assays for evaluating active immunization, and Example 5 describes the evaluation of a chimeric protein of the presently described subject matter.

### Example 1

### Synthesis of Chimeric Proteins

The chimeric proteins of the presently described subject matter were produced using standard 15 recombinant methods. Initially, a gene-synthesis company, (Blue Heron Biotechnology Inc.) was contracted to make the initial plasmid based on the chimeric protein amino acid sequences described herein that were optimized for E. coli preferred codon usage. Briefly, the native NTHi pilin protein sequence was modified by truncating the N-terminus (residues 1-39 of SEQ ID NO: 2) and adding a HIS-tag sequence and a thrombin cleavage site as set out in SEQ ID NO: 3. The HIS-tag was preceded by a sequence (MUSS) to assist in expression. The thrombin cleavage site allowed for release of the HIS-tag. These plasmids were then cloned into the E. coli expression vector pET-15b vector (Novagen). The plasmid were then transformed into E. coli strain “Origami(DE3)” (available from Novagen) as the host for expression of soluble His-tagged chimeric proteins. Another E. coli host cell expression stain that may be used is Origami B(DE3) (Novagen).

The His-tagged variants of the chimeric proteins will be recovered by nickel column chromatography, then used for initial studies to determine if they are reactive with antisera directed against any of the following: native OMP P5-fimbrin, LB1 (full length 40 amino acid peptide), LB1(1) (a synthetic peptide representing just the 19 amino acid B-cell epitope of LB1), recombinant PilA protein or native PilA protein. Once the His-tag is removed by thrombin site cleavage, the recombinant huneric proteins will be used as immunogens to determine their immunogenicity and protective capability.

Exemplary chimeric proteins of the presently described subject matter have the sequences as set out in Table 2 below. The chimeric proteins having the amino acid sequences of SEQ ID NOS: 10, 12 and 14 have been expressed by E. coli as described above.

TABLE 2

| SEQ ID | |
|---|---|
| NO: | Chimeric Protein Amino Acid Sequence |
| 9 | MGSSHHHHHSSGLVPRGSHMTKKAAVSELLQASAPYKADVE |
| | LCRSDYKFYEDANGTRDHKKGCTGGKNGIAADITTAKGYVKS |
| | VTSNGAITVKGDGTLANMEYILQATGNAATGVTWTTCKGTDA |
| | SLFPANFCGSVTQ |
| 10 | MGSSHHHHHSSGLVPRGSHMTKKAAVSELLQASAPYKADVE |
| | LCLVRSDYKFYEDANGTRDHKKGHTCTGGKNGIAADITTAKG |
| | YVKSVTTSNGAITVKGDGTLANMEYILQATGNAATGVTWTTC |
| | KGTDASLFPANFCGSVTQ |
| 11 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCRSDYKFYEDANGTR |
| | DHKKGCGSVTQ |
| 12 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCLVRSDYKFYEDANGT |
| | RDHKKGRHTCGSVTQ |
| 13 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCKGTDASLFPANFCGS |
| | VTQRSDYKFYEDANGTRDHKKG |
| 14 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCKGTDASLFPANFCGS |
| | VTQLVRSDYKFYEDANGTRDHKKGRHT |
| 15 | MGSSHHHHHSSGLVPRGSHMTKKAAVSELLQASAPYKADVE |
| | LCRSDYKLYNKNSSSNSTLKNLGECTGGKNGIAADITTAKGYV |
| | KSVTTSNGAITVKGDGTLANMEYILQATGNAATGVTWTTCKG |
| | TDASLFPANFCGSVTQ |
| 16 | MGSSHHHHHSSGLVPRGSHMTKKAAVSELLQASAPYKADVE |
| | LCLVRSDYKLYNKNSSSNSTLKNLGERHTCTGGKNGIAADITT |
| | AKGYVKSVTTSNGAITVKGDGTLANMEYILQATGNAATGVTW |
| | TTCKGTDASLFPANFCGSVTQ |
| 17 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYTLQATGNAATGVTWTTCRSDYKLYNKNSSSN |
| | STLKNLGECGSVTQ |
| 18 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCLVRSDYKLYNKNSSS |
| | NSTLKNLGERHTCGSVTQ |
| 19 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCKGTDASLFPANFCGS |
| | VTQRSDYKLYNKNSSSNSTLKNLGE |
| 20 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCKGTDASLFPANFCGS |
| | VTQLVRSDYKLYNKNSSSNSTLKNLGERHT |
| 21 | MGSSHHHHHSSGLVPRGSHMTKKAAVSELLQASAPYKADVE |
| | LCRSDYKLYNKNSSSLKNLGECTGGKNGIAADITTAKGYVKSV |
| | TTSNGAITVKGDGTLANMEYILQATGNAATGVTWTTCKGTDA |
| | SLFPANFCGSVTQ |
| 22 | MGSSHHHHHSSGLVPRGSHMTKKAAVSELLQASAPYKADVE |
| | LCLVRSDYKLYNKNSSSTLKNLGERHTCTGGKNGIAADITTAK |
| | GYVKSVTTSNGAITVKGDGTLANMEYILQATGNAATGVTWTT |
| | CKGTDASLFPANFCGSVTQ |
| 23 | MGSSHHHHHSSGLVPRGSHMTKKAAVSELLQASAPYKADVE |
| | LVTSTNETTNCTGGKNGIAADITTAKGYVKSVTTSNGAITVKG |
| | DGTLANMEYILQATGNAATGVTWTTCRSDYKLYNKNSSTLKN |
| | LGECGSVTQ |
| 24 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCLVRSDYKLYNKNSST |
| | LKNLGERHTCGSVTQ |
| 25 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCKGTDASLFPANFCGS |
| | VTQRSDYKLYNKNSSTLKNLGE |
| 26 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCKGTDASLFPANFCGS |
| | VTQLVRSDYKLYNKNSSTLKNLGERHT |
| 27 | MGSSHHHHHSSGLVPRGSHMTKKAAVSELLQASAPYKADVE |
| | LCRSDYKFYDNKRIDCTGGKNGIAADITTAKGYVKSVTTSNGA |
| | ITVKGDGTLANMEYILQATGNAATGVTWTTCKGTDASLFPANF |
| | CGSVTQ |
| 28 | MGSSHHHHHSSGLVPRGSHMTKKAAVSELLQASAPYKADVE |
| | LCLVRSDYKFYDNKRIDRHTCTGGKNGIAADITTAKGYVKSVT |
| | TSNGAITVKGDGTLANMEYILQATGNAATGVTWTTCKGTDAS |
| | LFPANFCGSVTQ |
| 29 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCRSDYKFYDNKRIDCG |
| | SVTQ |
| 30 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCLVRSDYKFYDNKRID |
| | RHTCGSVTQ |
| 31 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCKGTDASLFPANFCGS |
| | VTQRSDYKFYDNKRID |
| 32 | MGSSHHHHHSSGLVPRGSHMTKKAAVSELLQASAPYKADVE |
| | LCVTSTNETTNCTGGKNGIAADITTAKGYVKSVTTSNGAITVK |
| | GDGTLANMEYILQATGNAATGVTWTTCKGTDASLFPANFCGS |
| | VTQLVRSDYKFYDNKRIDRHT |

Additional exemplary chimeric proteins of the presently described subject matter have the amino acid sequences as set forth in Table 3 below. These chimeric proteins have been expressed by E. coli and purified using a HIS-tag, as described above. The chimeric proteins set out in Table 3 have the His tag sequence removed for use as an immunogen. The chimeric protein having the amino acid sequence of SEQ ID NO: 56 was used in the studies described in Example 5.

TABLE 3

| SEQ ID | |
|---|---|
| NO: | Chimeric Protein Amino Acid Sequence |
| 54 | GSHMTKKAAVSELLQASAPYKADVELCLVRSDYKFYEDANGT |
| | RDHKKGRHTCTGGKNGIAADITTAKGYVKSVTTSNGAITVKGD |
| | GTLANMEYILQATGNAATGVTWTTTCKGTDASLFPANFCGSV |
| | TQ |
| 55 | GSHMTKKAAVSELLQASAPYKADVELCVYSTNETTNCTGGKN |
| | GIAADITTAKGYVKSVTTSNGAITVKGDGTLANMEYILQATGNA |
| | ATGVTWTTTCLVRSDYKFYEDANGTRDHKKGRHTCGSVTQ |
| 56 | GSHMTKKAAVSELLQASAPYKADVELCVYSTNETTNCTGGKN |
| | GIAADITTAKGYVKSVTTSNGAITVKGDGTLANMEYILQATGNA |
| | ATGVTWTTTCKGTDASLFPANFCGSVTQLVRSDYKFYEDAN |
| | GTRDHKKGRHT |
| 57 | GSHMLVRSDYKFYEDANGTRDHKKGRHTGPSLKLTKKAAVS |
| | ELLQASAPYKADVELCVYSTNETTNCTGGKNGIAADITTAKGY |
| | VKSVTTSNGAITVKGDGTLANMEYILQATGNAATGVTWTTTC |
| | KGTDASLFPANFCGSVTQ |

### Example 2

### Immunogenicity of Chimeric Proteins

Rabbits or chinchillas are immunized with the chimeric proteins. Rabbits receive an initial immunizing dose of 500 μg of a chimeric protein in complete Freund's adjuvant. The rabbits receive a second dose of 400 μg of the chimeric protein 21 days later. The rabbits receive a third dose of chimeric protein in complete Freund's adjuvant 42 days after the initial immunizing dose with 400 μg of the same peptide in either IPA or PBS (one rabbit per diluent). Sera are obtained 3 weeks after each dose. Chinchillas receive an initial immunizing dose of 10 μg of the chimeric protein in the adjuvant monophosphoryl lipid A (MPL). One month (˜30 days) later, chinchillas receive a second identical dose. The third and final dose is delivered 30 days after the second dose. Sera are obtained ˜10-14 days after each dose. The sera from all animals are assessed for titer and specificity against the LB1 peptide (40-mer), LB1(1), PilA protein and the chimeric proteins, by ELISA, Western blot and biosensor. Antisera are also tested against whole bacteria via flow cytometry (FACS) analysis.

### Example 3

### Evaluating Passive Immunization

The protection conferred by an animal's immune response directed against the chimeric proteins of the invention is determined in a chinchilla model of experimental otitis media. Chinchillas are passively immunized with 5 ml/kg hyperimmune chinchilla or human serum directed against a chimeric protein of the invention. Control chinchillas receive normal chinchilla serum or normal human serum. Next the chinchillas receive first a viral co-pathogen intranasally, then a week later, an intranasal challenge with the NTHi bacteria. The chinchillas are examined and rated daily or every 2 days for up to 35 days after bacterial challenge. Immunized chinchillas receiving immune chinchilla or human serum display reduced tympanic membrane pathology and reduced or absent signs of infection of the middle ear space as determined by both video otoscopy and tympanometry. In this assay, the presence of middle ear fluids in chinchillas receiving chinchilla or human anti-chimeric protein serum is reduced when compared to controls.

### Example 4

### Evaluating Active Immunization

Cohorts of 5-10 chinchillas each are actively immunized with either a saline control preparation, an adjuvant-only preparation, or one of the chimeric proteins of the invention that has been admixed with an appropriate adjuvant. The immunogens are assessed for endotoxin content prior to their use as an immunogen via a chromogenic Amoebocyte Lysate assay which is commercially available from Whittaker Bioproducts under the designation QCL-1000. The chinchillas are then subcutaneously injected with 10 μg immunogen in the adjuvant MPL (or another appropriate adjuvant). Then 30 days later they receive 10 μg of the same immunogen in MPL. Thirty days following the second immunization, these animals receive the final immunizing dose. Approximately 10-14 days after the final immunizing dose is delivered, chinchillas are challenged both transbullarly and intranasally with a strain of NTHi. The chinchillas are assessed over a 35-day period for: tympanic membrane pathology by video otoscopic examination and tympanometry; semiquantitation of NTHi recovered via epitympanic tap of the inferior bulla and passive lavage of the nasopharynx; and light microscopic examination of fixed middle ear mucosal epithelium and tympanic membrane for histopathology. For example, chinchillas immunized with the chimeric proteins of the invention will have reduced tympanic membrane pathology, will be free of middle ears effusions or they will contain effusions that are culture-negative, there will be reduced or no biofilm present in the tympanum and there will be minimal thickening of the middle ear mucosa, minimal osteoneogensis and reduced presence of both red blood cells and inflammatory cells in the subepithelial space.

### Example 5

### Evaluation of Chimeric Proteins

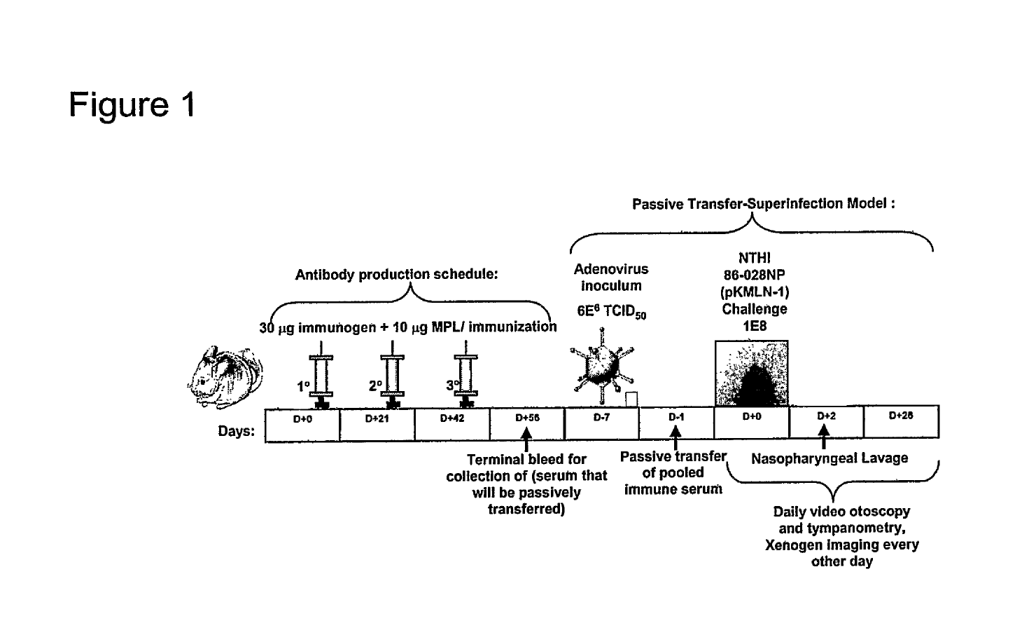

The protective efficacy of the chimeric protein having the amino acid sequence of SEQ ID NO: 56 (referred to as “chim-V3” herein) was evaluated using the chinchilla passive-transfer, superinfection model of OM. This chimeric peptide comprised the B-cell epitope of the LB1 peptide (SEQ ID NO: 5) expressed after the C-terminal glutamine residue of recombinant PilA (residues 40-149 of SEQ ID NO: 2). To generate polyclonal antiserum for use in passive transfer efficacy studies, the chim-V3 protein was delivered to a cohort of adult chinchillas with the adjuvant, monophosphoryl lipid A (MPL) plus trehalose dimycolate (Corixa). A timeline depicting the immunization regimen is set out in FIG. 1. To generate immune serum pools, alert prone chinchillas were immunized subcutaneously 3 times with 30 Kg of chim-V3 plus 10 μg of MPL or 10 μg MPL alone every 21 days. At day 56, a terminal bleed of the inoculated animals was collected and serum was pooled for transfer to naive juvenile animals. To study efficacy, a separate cohort of juvenile chinchillas was first challenged with adenovirus on day -7. Six days later (day -1), the pooled anti-chim-V3 immune serum was passively transferred to these adenovirus-compromised animals. The following day (day 0), animals that received anti-chim-V3 serum by passive transfer were challenged with the bacterium, nontypeable Haemmophilus influenzae. These animals were then monitored for incidence and severity of disease over a 26-day time-course (relative to bacterial challenge) by daily video otoscopy and tympanometry as well as Xenogen in vivo imaging every other day.

The titer of anti-chim-V3 antibody was measured in the immune serum collected from the inoculated animals using an ELISA. This analysis demonstrated that the collected antiserum contained antibodies specific for the chim-V3 protein. The presence of anti-chim-V3 antibodies in the collected antiserum as also confirmed using Western blot analysis.

FACS analysis was used to measure the ability of serum immunoglobulins from immunized animals to recognize surface exposed native structures expressed by NTHi 86-028 NP. NTHi bacteria were incubated with chim-V3 antiserum, washed, then incubated with naïve or immune FITC-Protein A, washed and analyzed by FACS analysis. Inoculation with the chim-V3 protein induced a significant increase in antibodies that were capable of recognizing the NTHi surface proteins or chim-V3 protein. The data obtained were dependent on both antibody titer and avidity as well as relative expression of both the type IV pilus and the OMP P5-homologous adhesion by NTHi when grown in vitro.

The luminescent reporter NTHi 86-028 NP pKMLN-1 was used to detect NTHi infection in the animals inoculated with chim-V3 protein using Xenogen in vivo real-time imagining. Growth curves of the luminescent strain NTHi 86-028 NP pKMLN-1 and the parental strain NTHi 86-028 demonstrated that growth of the luminescent NTHi strain was comparable to the parental strain. Luminescent imaging of NTHi residing in the nasopharynx of the inoculated animals was readily accomplished however, due to the microaerophilic nature of the diseased middle ear, luminescence of NTHi present in the middle ear could not be monitored over the entire disease course because the luminescence is dependent on the availability of oxygen. Animals were monitored every other day for the presence of luminescent bacteria, and if bacteria were detected, this was recorded as a luminescent event. Luminescent infection was detected at least six days after challenge in the inoculated animals. The total number of luminescent events in the chim-V3 inoculated animals was less than the total number of luminescent events in the control animals (inoculated with MPL only).

Throughout the course of the study, daily video otoscopy and tympanometry was used to determine the percent of chinchilla middle ears with OM. Inoculation with chim-V3 caused 53% reduction in the number of animals with middle ears having OM as compared to control animals (inoculated with MPL only).

All of these studies demonstrate that the chim-V3 protein was immunogenic and anti-chim-V3 antibodies were protective in the chinchilla passive transfer-superinfection model of OM.

All publications cited in the specification are indicative of the level of skill of those skilled in the art to which the presently described subject matter pertains. All of these publications are hereby incorporated by reference herein to the same extent as if each individual publication were specifically and individually indicated as being incorporated by reference.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

What is claimed:

1. A topical delivery system, comprising:
a topical immunogenic composition comprising at least one active agent comprising a chimeric protein comprising residues 40-149 of SEQ ID NO: 2 and SEQ ID NO: 4, SEQ ID NO: 4 being inserted before residue 40 of SEQ ID NO: 2; and
a flexible substrate in communication with the topical immunogenic composition.

2. The topical delivery system according to claim 1, wherein the topical immunogenic composition further comprises at least one pharmaceutically acceptable topical carrier.

4. The topical delivery system according to claim 1, wherein the active agent comprises the amino acid sequence of SEQ ID NO: 57.

5. A topical delivery system, comprising:
a topical immunogenic composition comprising at least one active agent comprising a chimeric protein comprising residues 40-149 of SEQ ID NO: 2 and SEQ ID NO: 4, SEQ ID NO: 4 being inserted before residue 40 of SEQ ID NO: 2.

6. A method for eliciting an immune response to NTHi bacteria, in a patient, comprising:
topically administering to the patient at risk of NTHi bacterial infection, a topical delivery system comprising a topical immunogenic composition comprising at least one active agent, the at least one active agent comprising an immunogenic dose of a chimeric protein comprising
an immunogenic PilA peptide selected from the group consisting of the amino acid sequence of SEQ ID NO: 6, SEQ ID NO: 7, SEQ ID NO: 8, and residues 40 to 149 of SEQ ID NO: 2, and
a portion of the LB1 peptide selected from the group consisting of the amino acid sequence of SEQ ID NO: 4 and SEQ ID NO: 5, wherein the LB1 peptide is inserted before the immunogenic PilA peptide.

7. The method according to claim 6, wherein the PilA peptide comprises residues 40-149 of SEQ ID NO: 2 and wherein the LB1 peptide comprises SEQ ID NO: 4.

8. The method according to claim 6, wherein the chimeric protein comprises the amino acid sequence of SEQ ID NO: 57.

9. The method according to 6, wherein administering comprises applying the topical delivery system at least once.

10. The method according to claim 6, wherein administering comprises applying the topical delivery system once a week, at weekly intervals, for a pre-determined period of time.

11. The method according to claim 10, wherein the predetermined period of time is from 2 weeks to 10 weeks.

12. The method according to claim 10, wherein the predetermined period of time is 2 weeks.

13. The method according to claim 6, wherein the topical delivery system remains on the patient for a period of at least 24 hours.

14. The method according to claim 6, wherein administering comprises applying the topical delivery system to an area of skin of the patient.

15. The method according to claim 6, wherein the topical delivery system further comprises a flexible substrate.

16. The method according to claim 6, wherein the NTHi infection is in the middle ear, nasopharynx or lower airway.

17. The method of claim 6, wherein the NTHi bacterial infection involves a pathological condition selected from the group consisting of otitis media, pneumonia, sinusitis, septicemia, endocarditis, epiglottitis, septic arthritis, meningitis, sepsis, salpingitis, epiglottis, pericardis, cellulitis, osteomyelitis, endocarditis, cholecystitis, intraabdominal infections, urinary tract infection, mastoiditis, aortic graft infection, conjunctitivitis, Brazilian purpuric fever, occult bacteremia, chronic obstructive pulmonary disease, chronic bronchitis, bronchietasis, and cystic fibrosis.

18. A method for treating a NTHi bacterial infection in a patient, comprising:
topically administering to the patient, a topical delivery system comprising a topical immunogenic composition comprising at least one active agent, the at least one active agent comprising an immunogenic dose of a chimeric protein comprising
an immunogenic PilA peptide selected from the group consisting of the amino acid sequence of SEQ ID NO: 6, SEQ ID NO: 7, SEQ ID NO: 8, and residues 40 to 149 of SEQ ID NO: 2, and
a portion of the LB1 peptide selected from the group consisting of the amino acid sequence of SEQ ID NO: 4 and SEQ ID NO: 5, wherein the LB1 peptide is inserted before the immunogenic PilA peptide.

19. The method of claim 18, wherein the patient suffers from at least one pathological condition selected from the group consisting of otitis media, pneumonia, sinusitis, septicemia, endocarditis, epiglottitis, septic arthritis, meningitis, sepsis, salpingitis, epiglottis, pericardis, cellulitis, osteomyelitis, endocarditis, cholecystitis, intraabdominal infections, urinary tract infection, mastoiditis, aortic graft infection, conjunctitivitis, Brazilian purpuric fever, occult bacteremia, chronic obstructive pulmonary disease, chronic bronchitis, bronchietasis, and cystic fibrosis.
